(19) United States
(12) Patent Application Publication
(10) Pub. No.: US 2008/0149110 A1
(43) Pub. Date: Jun. 26, 2008

(54) Awake airway, including two variants

(76) Inventor: Charles Craig Baldwin, Columbia, MO (US)

Correspondence Address: CHARLES CRAIG BALDWIN, 5000 DUSTIN COURT, COLUMBIA, MO, 65203, US

(21) Appl. No.: 11/602,918

(22) Filed: Dec. 22, 2006

(51) Int. Cl. A61F 5/37

(52) U.S. Cl. 128/846

(57) ABSTRACT

The Awake Airway is an oral device designed with two arches to gently hold both the maxillary and mandibular dental arches in a fixed position angled slightly open in such a way as to not stimulate any part of the oral cavity or oropharynx so a fully wide awake user would tolerate it. It would be made of a resilient material such as a soft plastic with a foam insert to hold the teeth or edentulous arches. It is shaped to conform to the teeth and maxillary and mandibular anatomy, and easily inserted by the user him/herself. By gently maintaining the mouth slightly open it would enhance the patency of the airway. It's usefulness would include that of a clinical airway in awake to sedated or anesthetized or obtunded patients that may be more flexible than currently available airway support devices since it may be tolerated until the user is fully awake. Another use might be in Obstructive Sleep Apnea (OSA) as the patient may place it themselves and tolerate it as they sleep with airway support that could supplement or replace current therapies. In the variant to anchor other airway devices a tie which might be anything from a ratcheted tie to a plain knotted tie can be molded into the incisor portion of the Maxillary arch or passed through an open provision for such a tie to firmly anchor a device such as an endotracheal tube, perhaps a more secure means than is currently utilized.

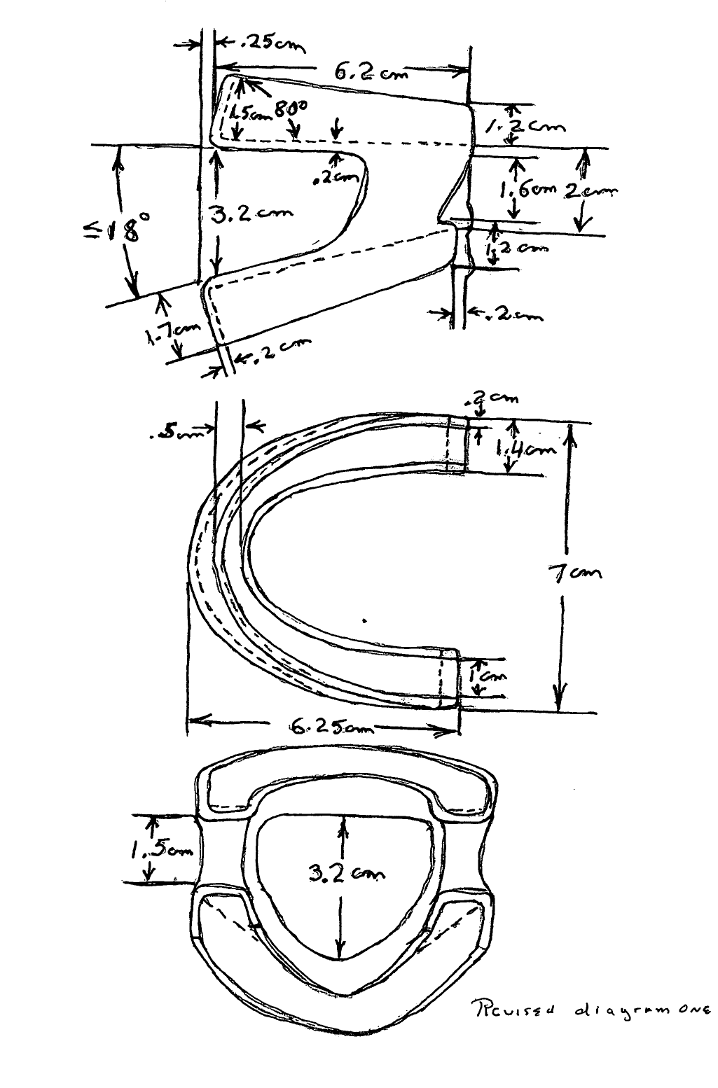

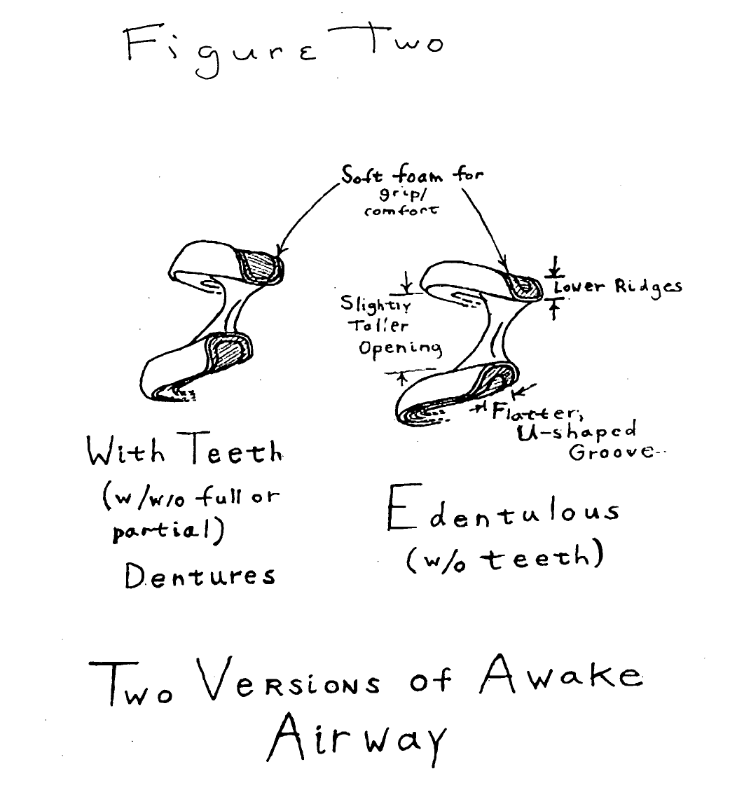

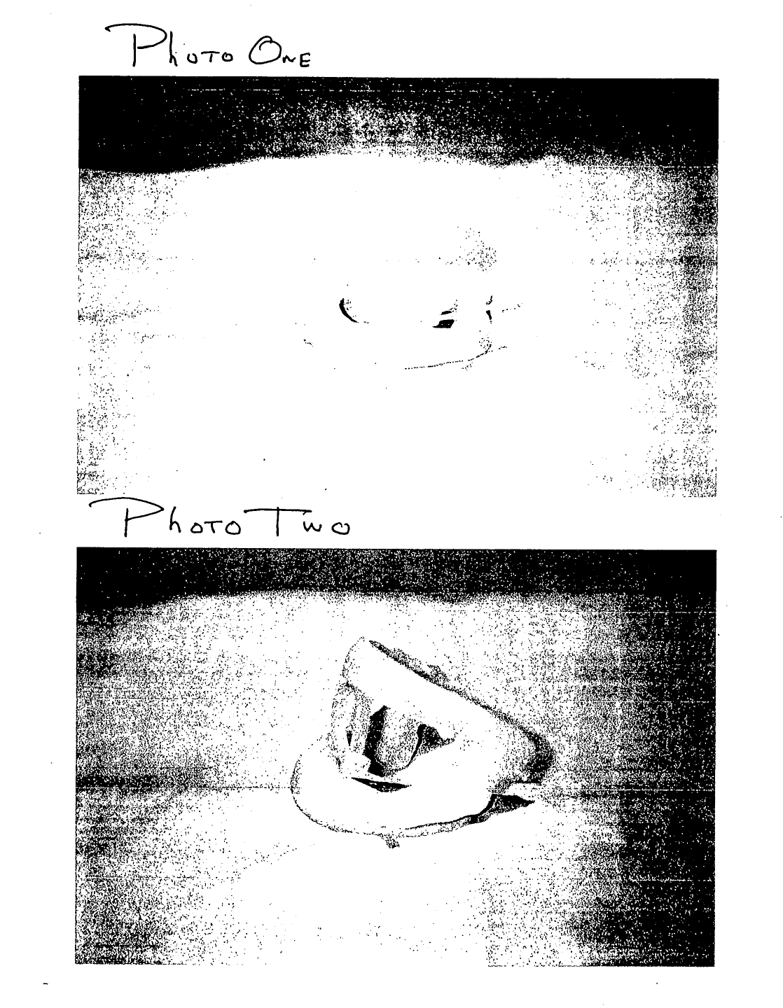

The Awake Airway is an oral device designed to fit on the dental arches or alveolar ridges of both the maxilla and mandible with connecting buttresses. The buttresses maintain an angular opening between the two arches, establishing an open or pharynx. The Airway is designed to be used in the awake person, a feature for which a utility patent application has been made. It has a jaw thrust feature, also a utility patent application, in which the mandibular component is moved a few millimeters frontally. This design exploit's the human temporomandibular joint's unique transition from a hinge joint to a cam joint, further opening the oropharynx without angling the mouth open any more. It is to come in two versions: one for those with teeth and/or dentures and the edentulous.

The Awake Airway may be used in the obtunded patient as well. A Variant proposed here has an anchoring device such zip or Nye tie to affix another airway device as such as an endotracheal tube or a laryngeal mask airway (LMA). The Awake Airway stabilizes the movable components of the human airway such as the mandible. By affixing an airway such as an endotracheal tube to it in a ventilated patient it may be more stable than taping it to the lip or using any other current method. This may be especially useful in the edentulous, in whom an endotracheal tube taped to the lip has a great deal of movement that can affect the ventilation of the patient.

### Linked Applications

The application is linked to three other utility applications and one design application.

The utility applications are:

1) An application for the nongagging nature of all versions and variants of the Awake Airway.

2) An application for the jaw thrust feature of the Awake Airway which exploits the unique function of the human temporomandibular joint as a cam joint when moved frontally to further open the oropharynx, enhancing the maintenance of a patent airway.

3) An application for a Glossal Variant which incorporates an arch across the mandibular component to hold and stabilize the tongue. This may be useful in the obese and those with a small mouth or large tongue, as well as others.

The design application is for the unique and readily recognizable shape and appearance of the Awake Airway, including the Anchor Variant of this application as well as of all other versions and variants.

### ATTACHMENTS TO THIS APPLICATION

Figure One: Design graphic for a large version of the Awake Airway.

Figure Two: Drawings of the two versions of the Awake Airway: the Toothed and Non-toothed (Edentulous) Versions

Photos One and Two: photos of the Awake Airway with an affixed zip tie as an anchor.

I claim

1. A parabolic arch shaped support to gently and firmly grasp the maxillary dental arch; the support covering from the incisor teeth to the molar teeth or, for the edentulous, the entire maxillary alveolar arch; the support comprised of a resilient and firm material molded into a cross-sectional u-shape; an outside (buccal) edge or lip and inside (lingual) edge or lip comprising the two sides of the cross sectional u-shape, with a softer foam material inserted inside the resilient material

2. a second support arch as claimed in claim 2 to also have a parabolic arch shaped to follow the mandible also having an insert of foam material in the cross-sectional u shape with a buccal and lingual lip and floor to gently hold the teeth or alveolar ridges with differing thicknesses to accommodate them

3. the floor or bottom of the cross sectional u shape as claimed in claim 1 for the maxillary arch and in claim 2 for the mandibular arch to be narrow at the peak of the arch to accommodate the incisor teeth, widening toward both legs of the arch, maximum at the edge to accommodate the molar teeth/or wider alveolar ridge.

4. an outside (buccal) lip and an inside (lingual) lip as claimed in claim 1 and in claim 2 to be angled a few degrees (circa 15) degrees) posteriorly from a vertical from the base of the support as claimed in claim one and claim two and to be sufficiently high to cover the incisor teeth at the apes of the arch.

5. an outside buccal lip and an inside (lingual) lip as claimed in claim 1 and in claim 2 to have the angle from the base of the support approach back to vertical as it is molded toward the molar teeth and to diminish in height to just cover the molar teeth (usually lower than the incisor teeth).

6. An inside (lingual) lip or edge as claimed in claims 4 and 5 to be shorter than the outside (buccal) lip or edge to better conform to the angle of the mucosa arising from the lingual edge of the incisor teeth.

7. An inside (lingual) lip or edge as claimed in claim 1 and claim 2 to taper more that the outside (buccal) lip or edge to accommodate the fuller and flatter angle of the lingual mucosa.

8. a buttress or arch molded in the maxillary support as claimed in claim 1 to be affixed to the mandibular arch support in claim 2 reversed 180 degrees from the maxillary support to gently grasp the mandibular dental arch so as to hold the two arches angled open from one another

9. the buttress in claim 8 to be molded into the molar portions of the maxillary and mandibular arches, extending forward toward the premolars, and molded not to extend beyond the edge of either the mandibular or maxillary arches so as to have no edges, just a smooth molded surface

10. a buttress or arch as claimed in claim 8 to have an oval to rounded rectangular cross sectional shape at all levels including its attachment to the underside of the alveolar arch support and to angle the two alveolar arch supports to obtain an opening of more than a centimeter and/or an angle of 15 degrees and maintain tern in that relationship.

11. two arch supports as claimed in claim 1 and claim 2 and a buttress or arch as claimed in claim 8 together to gently mold and grasp the maxilla and mandible, whether with teeth or edentulous, and keep the mouth gently but firmly opened without touching any oral or pharyngeal structures that would gag or stimulate the wearer

12. a buttress or arch as claimed in claim 8 and claim 11 that gently advances the mandibular arch forward (frontally) sufficiently to engage the cam joint mechanics of the human Temporo-mandibular joint to open the oropharynx and maintain patency of the airway

13. a buttress or arch as claimed in claim 8 and 11 to have a smooth signoid shape from the maxillary to mandibular supports, thereby holding an open angle between the two arches of approximately 15 degrees and a frontal positioning of the mandibular arch vis-à-vis the maxillary arch to position the mandibular condyle over the meniscus of the temporomandulbar joint exploiting its cam mechanics.

14. on the underside of the incisor portion of the maxillary arch as claimed in claim 1 an attachment is molded into the resilient material to accommodate an encircling or tying device.

15. the attachment as claimed in claim 14 might incorporate the tie made of the same material such as a ratcheted or zip tie or have a patent channel molded in to accommodate such a device or an actuall tie such as umbilical tape that could be knotted around another airway device.

16. the attachment as claimed in claim 15 would firmly affix such other devices to the maxillary arch to stabilize it's relationship to other tissues such as the pharynx or trachea.
